(19) United States
(12) Patent Application Publication
(10) Pub. No.: US 2009/0124584 A1
(43) Pub. Date: May 14, 2009

(54) REGENERATION OF VAGINAL TISSUE WITH NON-SYSTEMIC VAGINAL ADMINISTRATION OF ESTROGEN

(75) Inventor: John W. Lyle, Vero Beach, FL (US)

Correspondence Address: DILWORTH & BARRESE, LLP, 333 EARLE OVINGTON BLVD., SUITE 702, UNIONDALE, NY, 11553, US

(73) Assignee: LYLE CORPORATE DEVELOPMENT, INC., Farmingdale, NJ (US)

(21) Appl. No.: 11/660,711

(22) PCT Filed: Jan. 20, 2006
(86) PCT No.: PCT/US2006/002145

§ 371 Date: Jan. 15, 2009

Related U.S. Application Data
(60) Provisional application No. 60/751,104, filed on Dec. 16, 2005.

(51) Int. Cl. A61K 31/565; A61K 31/56; C12Q 1/02

(52) U.S. Cl. 514/170; 514/182; 435/29

(57) ABSTRACT

Methods and formulations for the regeneration of vaginal tissue and vaginal cell health resulting from vaginal cell hypoxia in a human female. A pharmaceutical composition for topical non-systemic administration is formulated containing a hormonal agent administered to the vagina, vulvar area of the individual undergoing treatment.

This application claims priority to U.S. provisional application No. 60/751,104, filed on Dec. 16, 2005, the contents of which are incorporated by reference herein.

### BACKGROUND OF THE INVENTION

This invention relates to compositions that are useful for the revitalization of devitalized vaginal tissue. More particularly, this invention relates to the non-systemic administration of an estrogen and/or androgen to the vaginal, vulvar and/or urethral area of a female patient, for the regeneration of vaginal tissue and vaginal cell health.

The etiology of vaginal pathologies may include vascular/endothelial disease such as hypertension and diabetes, neurological disorders, hormonal disorders, such as decreased levels of estrogen and/or testosterone, medical therapies, such as, chemotherapy and radiation. For example, the commonly reported sexual problem in women with diabetes mellitus is lack of vaginal lubrication. It has been postulated that reduced vaginal lubrication in diabetic women may result from the structural changes of the vagina and vaginal tissue cells.

The vasculature and microvasculature of the vagina are innervated by nerves containing neuropeptides and other neurotransmitters, and upon stimulation vaginal engorgement enables transudation to occur and this process is responsible for increased vaginal lubrication. Transudation allows a flow of plasma through the epithelium and onto the vaginal surface, the driving force for which is increased blood flow in the vaginal capillary bed during the aroused state. In order for transudation to take place a sufficient number of healthy vaginal cells are necessary for a female sexual arousal response relating to genital (e.g. vaginal and clitoral) blood flow. Inadequate genital response to sexual stimulation may be due to lack of vagina and/or the clitoral engorgement that results from illnesses, which cause damage to the vascular structures that supply the vaginal tissues. Such illnesses are, for example, diabetes, hypertension and atherosclerosis, or the result of medical treatments, such as, radiation or chemotherapy, all of which affect the health and vitality of vaginal tissue cells.

In women, diabetes can lead to hardening of the blood vessels of the vaginal wall. Decreased blood flow due to diabetes may cause the vagina to be too dry, both normally and during arousal. It also may cause a woman to be at much greater risk of getting recurring yeast infections.

Atrophic vaginitis is an inflammation of the vagina due to thinning and shrinking tissues and decreased lubrication of the vaginal walls. It is caused by a lack of estrogen and a decrease in blood supply and nutrients to the vaginal tissue, and those two changes together can cause the vaginal wall to thin out. Low levels of estrogen in women will also cause the vagina to become less acidic which may cause changes to the vaginal flora, so the vagina becomes more prone to trauma and infection.

Current medical practices for the treatment of menopausal, diabetic woman and atrophic vaginitis includes estrogen replacement therapy (ERT), which is important in keeping blood flowing in vaginal tissues. Estrogen also provides an acid level adequate to protect against vaginal infections in women past menopause.

ERT does pose risks for some women. When hormone replacement therapy (i.e. estrogen and/or progestin to replace the hormones lost at menopause) is administered systemically to relieve hot flashes, night sweats, and vaginal dryness and to improve women's health, increased risk in breast cancer, heart attacks, strokes, and blood clots have been reported. The associated systemic administration of progesterone and/or testosterone may produce side effects such as, masculinisation with acne and excess body hair, scalp hair loss, fluid retention, deepening of the voice, enlargement of the clitoris and adverse effects on blood cholesterol.

Women undergoing chemotherapy or radiotherapy for malignant diseases such as leukemia often experience vaginal dryness as a result of treatment. Many disease states, such as systemic sclerosis and other systemic autoimmune disorders, Ehlers-Danlos syndrome, diabetes mellitus, and Sjogren's syndrome have decreased vaginal hydration and lubrication problems as significant disease-associated symptoms.

Vaginal tissue cells may become hypoxic because of limited diffusion of oxygen from blood vessels, or they can be acutely or transiently hypoxic because of intermittent blood flow. Vaginal tissue cells exposed to radiation and/or chemotherapy will experience detrimental effects and like all cells deprived of oxygen and nutrients they ultimately die. Because of the interdependence of all cells and their vasculature, prolonged ischemia secondary to blood vessel function will result in cell necrosis.

The present invention is advantageous as it provides a means for regenerating vaginal tissue-namely increased genital blood flow leading to vaginal, clitoral and labial engorgement through the improvement of the overall health of female vaginal tissue. Thus, the present invention provides a means to restore, or potentiate, the normal vaginal tissue through improved vaginal cell health as determined by vaginal tissue blood flow, regeneration of vaginal cells, and increasing the number of healthy vaginal cells present.

### SUMMARY OF THE INVENTION

The present invention provides a method of treating a human female exhibiting vaginal cell hypoxia and who is not currently receiving chronic hormonal therapy, the method comprising topically administering to the vaginal tissue of the female patient at least once in a seven day period a non-systemic vaginal cell hypoxia treatment amount of a composition comprising at least one hormone selected from the group consisting of estrogen and androgen and/or pharmaceutically acceptable salt and ester thereof.

In another embodiment of the present invention, a method of treating vaginal tissue in a human female undergoing or having undergone, a therapy or treatment that impairs the health of vaginal tissue cells and who is not currently receiving chronic hormonal therapy, the method comprising topically administering to the vaginal tissue of the female patient, at least once in a seven day period a non-systemic vaginal cell hypoxia treatment amount of a composition comprising at least one hormone selected from the group consisting of estrogen and androgen and/or pharmaceutically acceptable salt and ester thereof.

In yet another embodiment of the present invention, a method of treating vaginal tissue in a human female exhibiting clinical signs of a disease or condition that impairs the health of vaginal tissue cells and who is not currently receiving chronic hormonal therapy, the method comprising topically administering to the vaginal tissue of the female patient, at least once a in a seven day period, a non-systemic vaginal cell hypoxia treatment amount of a composition comprising at least one hormone selected from the group consisting of estrogen and androgen and/or pharmaceutically acceptable salt and ester thereof.

In describing and claiming the present invention, the following terms and expressions shall be understood to have the designated meanings.

“Administration,” and “administering” refer to the manner in which a drug is presented to a subject. Administration can be accomplished by various routes well-known in the art, however, as contemplated herein, non-oral methods, such as topical application is the preferred method.

“Androgenic steroid,” or “androgen,” refer to a steroid, natural or synthetic, which exerts its biological or pharmacological action primarily by binding to androgen receptors. Examples include, but are not limited to: testosterone, methyltestosterone, androstenedione, adrenosterone, dehydroepiandrosterone, oxymetholone, fluoxymesterone, methandrostenolone, testolactone, pregnenolone, 17α-methylnortestosterone, norethandrolone, dihydrotestosterone, danazol, androsterone, nandrolone, stanozolol, ethylestrenol, oxandrolone, bolasterone, mesterolone, testosterone propionate, testosterone cypionate, testosterone phenylacetate, and testosterone enanthate, testosterone acetate, testosterone buciclate, testosterone heptanoate, testosterone decanoate, testosterone caprate, testosterone isocaprate, as well as esters, derivatives, prodrugs, and isomers thereof.

The term “atrophy” refers to the diminution in the size of a cell, tissue, or organ from its fully developed normal size. Atrophy is a general physiological process of reabsorption and breakdown of tissues, involving apoptosis on a cellular level. It can be part of normal body development and homeostatic processes, or as a result of disease. Pathological atrophy refers to atrophy resulting from disease of the tissue itself, or loss of trophic support due to other disease. Atrophy may also be defined as a wasting or decrease in the size of an organ or tissue, as from death and reabsorption of cells, diminished cellular proliferation, pressure, ischemia, malnutrition, decreased function, or hormonal changes.

The term “chemotherapy” as used herein is the treatment of cancer using specific chemical agents or drugs that are selectively destructive to malignant cells and tissues. Chemotherapy uses generally selectively toxic substances, i.e., substances that can destroy or inhibit malignant tissue. Chemotherapeutic substances differentially affect biochemical reactions in different tissues; e.g. antimetabolites which are more toxic to rapidly proliferating cells that, include healthy cells as well as those associated with cancer.

The term “clinical” as defined herein refers to the direct observation of a patient, or to the course of a disease of a patient and the observed symptoms of a patient for the determination of a clinical diagnosis of that patient.

“Coadministration” and similar terms refer to administration of multiple substances to one individual, either simultaneously or sequentially. Thus, with reference to estrogen and androgen, the term includes any situation in which women are receiving non-oral estrogen and non-oral androgen.

“Devitalized tissue” and “devitalized cells” as use herein describes vaginal tissue or cells that have lost their vitality or have been diminished or destroyed. Devitalized tissue may also be called necrotic or dead tissue which exhibits a characteristic gray appearance, due to a lack of oxygen and nutrients. The cause of devitalized or necrotic cells or tissues is through injury, illness, disease, especially in a localized area of the body, chemotherapy and radiation therapy.

The term “disease” as used herein is an actual physical, pathophysiological process that can cause an abnormal condition of the body or mind. A pathological condition of a part, organ, or system of an organism resulting from various causes which include, but are not limited to infection, genetic defect, or environmental stress, and characterized by an identifiable group of signs or symptoms. Disease may impair the normal state or functioning of the body as a whole or of any of its parts.

The term “effective amount” is used throughout the specification to describe concentrations or amounts of compounds according to the present invention which may be used to produce a favorable change in the symptomology, disease or condition treated, whether that change is a decrease in or reversal of the effects of symptomology or disease state depending upon the disease state or condition treated. In the present invention, in preferred aspects, an effective amount is that amount which is used to treat the pathology and symptomology associated with vaginal tissue hypoxia. An effective amount for purposes of treating one or more symptoms of the present invention, includes the non-systemic manner in which an active compound is administered to a patient.

“Estrogen,” and “estrogenic hormone” refer to any substance, natural or synthetic, that exerts a biological or pharmacological action primarily by binding to estrogen receptors. Examples include but are not limited to: 17-β-estradiol, 17-α-estradiol, estriol, estrone, and phytoestrogens. These estrogens may be derivatized or modified to form, for example, conjugated equine estrogens, esterified estrogens, ethinyl estradiol, etc. Examples of esterified estrogens include but are not limited to: estradiol-3,17-diacetate, estradiol-3-acetate, estradiol-17-acetate, estradiol-3,17-divalerate, estradiol-3-valerate, estradiol-17-valerate. The estrogens may also be present as salts, (e.g., as sodium estrogen sulfate), isomers, or prodrugs.

Also included, are phytoestrogens which are plant-derived estrogens. Isoflavones are one major form of phytoestrogen and have a common diphenolic structure that resembles the structure of potent synthetic estrogens such as diethylstilbesterol and hexestrol. Major isoflavones found in humans include, but are not limited to genistein, diadzein, and equol.

The terms “formulation” and “composition” are used interchangeably herein. The terms “pharmaceutical” and “drug” are also used interchangeably to refer to a pharmacologically active substance or composition. These terms of art are well-known in the pharmaceutical and medicinal arts.

The term “health” as defined herein is physical and mental wellness, a healthy state of wellbeing free from disease and absence of illness, or functionally, as the ability to cope with everyday activities. In living organisms, health is a form of homeostasis, which is a state of balance, with inputs and outputs of energy and matter in equilibrium (allowing for growth).

The term “hypoxia” as used herein describes a deficiency in the amount of oxygen reaching body tissues or a condition of insufficient levels of oxygen in tissue or blood. Hypoxia at a cellular level develops when delivery of oxygen to cell mitochondria slows as the partial pressure gradient from capillaries to tissues decreases. As the delivery of oxygen decreases aerobic metabolism stops and less efficient anaerobic pathways of glycolysis become responsible for the production of cellular energy. The end result is an increase in cellular concentrations of sodium, calcium and hydrogen ions which lead to cell death.

The term “illness” as used herein is an impairment of normal physiological function affecting part or all of an organism. Illness can be a synonym for disease or it can be a person's perception of having poor health. Illness and disease are not necessarily the same.

“Improved vaginal cell health” refers to reducing, improving, or preventing the incidence of pathology associated with estrogenic or androgenic steroid deficiency of the vaginal tissue. Examples of such pathologies include but are not limited to: thinning of the vaginal wall; deceased numbers of vaginal cells; decreased blood flow to vaginal tissue, e.g. generalized vaginal cell hypoxia and/or ischemia, which may or may not be the result of an illness, disease and/or medical treatment, such as, chemotherapy or radiation therapy.

Physiological evaluations may be employed for measuring the achievement of desired effects in the case of androgen and estrogen delivery to the vaginal cells, which are well known in the art. Such evaluations may be performed by a physician, or other qualified medical personnel, and may include physical examination, blood tests, tissue samples and histological examination.

“Local administration” means administration by a non-systemic route at or in the vicinity of the site of an affliction, disorder or complication.

The term “menopause” is used throughout the specification to describe the period in a woman's life between the ages of approximately 45 and 50 after which menstruation (menses) naturally ceases. The symptomology associated with menopause which is particularly relevant to the present invention includes bone loss associated with osteoporosis and most importantly, vaginal dyspareunia.

The term “non-systemic” refers to local administration such that the compound being administered does not significantly enter the blood stream.

“Radiation therapy” or “radiotherapy” as used herein is high-energy rays used to damage cancer cells and stop them from growing and dividing. Radiation therapy can cause inflammation of tissues and organs in and around the body site radiated. This can cause damage to organs and circulating blood cells affecting the general heath and vitality of the individual receiving the treatment.

By “systemic” administration is meant oral, intravenous, intraperitoneal and intramuscular administration of a compound that provides a significant blood level.

“Testosterone” refers to the compound having the IUPAC names (17 β)-17-Hydroxyandrost-4-en-3-one, and Δ4-androsten-17β-ol-3-one, as well as their isomers. Testosterone is listed in the Merck Index, entry no. 9322, at page 1569, 12th ed., (1996).

“Therapeutic effect” refers to a desired result which is achieved to some degree. In the context of estrogen and androgen supplementation of the present patent application, the desired results are referred to as “regeneration of vaginal tissue,” or revitalization of vaginal tissue.” In one aspect, therapeutic effects may be achieved by delivering a non-systemic “effective amount” of a substance capable of achieving the desired result to a selected degree. While the achievement of therapeutic effects may be measured by a physician or other qualified medical personnel using evaluations known in the art, it is recognized that individual variation and response to treatments may make the achievement of therapeutic effects a subjective decision.

The term “therapy” as defined herein is the systematic application of remedies to effect a cure or a treatment intended to cure or alleviate an illness or injury, whether physical or mental. Examples of therapies or treatments are drug and/or chemotherapy, radiotherapy, immunosuppressive therapy, and the like.

By “topical administration” is meant non-systemic administration and includes the application of the compounds of the invention externally to the epidermis and instillation of such a compound into and around the vagina and vaginal area (e.g., the individual anatomical parts, such as, labia majora, labia minora, clitoris, etc.) of a female, and where it does not significantly enter the blood stream. As contemplated herein, topical application is the preferred method.

The term “vaginal dyspareunia” is used throughout the specification to describe a symptom or condition of menopause wherein vaginal atrophy, dryness and pain during sexual intercourse occurs.

“Vaginal application” means the administration of a composition directly to the vaginal skin surface and from which an effective amount of drug is released. Examples of topical formulations include but are not limited to ointments, creams, gels, sprays, vaginal rings, and pastes.

Vaginal administration can be accomplished by applying, pasting, rolling, attaching, pouring, pressing, rubbing, etc., of a topical preparation onto the vaginal skin surface. These and additional methods of administration are well-known in the art.

“Woman” refers to a human female who benefits from an androgen or estrogen administration in any way. In one aspect, the female may be pre, peri or post menopausal due to age, oophorectomy, or ovarian failure. In yet another aspect, the female may display a deficiency, or imbalance of the vaginal cells which may benefit from the topical non-systemic administration of an estrogen and/or androgenic hormone.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

### DETAILED DESCRIPTION

The present invention provides for a method of treating a human female exhibiting vaginal cell hypoxia and who is not currently receiving chronic hormonal therapy, the method comprising topically administering to the vaginal tissue of the female patient at least once in a seven day period a non-systemic vaginal cell hypoxia treatment amount of a composition comprising at least one hormone selected from the group consisting of estrogen and androgen and/or pharmaceutically acceptable salt and ester thereof.

The present invention further provides methods of treating vaginal tissue in human females undergoing or having undergone chemotherapy and/or radiotherapy, or displaying diseases or illnesses such as diabetes, hypertension, atherosclerosis, and like conditions which impair the normal health of vaginal tissue cells, and who are not currently receiving chronic hormonal therapy. The methods comprise topically administering directly to the vaginal tissue of the female patient, at least once in a seven day period a non-systemic vaginal cell hypoxia treatment amount of at least one hormone selected from the group consisting of an estrogen, androgen and pharmaceutically acceptable salt, ester or pro-drug thereof. The method of treating or regenerating vaginal tissue alleviates the symptoms of vaginal cell hypoxia and/or atrophy caused by the illnesses, diseases and/or therapies, discussed supra, by increasing the flow of blood and nutrients to vaginal tissue cells.

The methods described herein are useful in female patients in need of such treatment resulting from vaginal cell hypoxia wherein the female patients are pre, peri, or post menopausal.

The pharmaceutical formulations of the invention will include at least one member selected from the group consisting of an estrogen and androgen or a pharmaceutically acceptable salt, ester, or pro-drug thereof. “Pharmaceutically acceptable salts, esters, or inclusion complexes” refer to those salts, esters and inclusion complexes which retain the biological effectiveness and properties of the base compounds and which are not biologically or otherwise undesirable.

Salts, esters and inclusion complexes of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from the free base (typically wherein the neutral form of the drug has a neutral NH₂ group) using conventional means, involving reaction with a suitable acid.

Estrogens will generally be selected from the group consisting of, pharmaceutically acceptable salts and esters of any of the foregoing, and mixtures thereof. Specifically, such steroids include estradiol, estradiol benzoate, estradiol cypionate, estradiol dipropionate, estriol, estrone, estrone benzoate, ethynyl estradiol, and mestranol, in the estrogen family, and acetoxypregnenolone, ethisterone, fluorogestone acetate. Additionally, with pharmaceutical formulations adapted for vulvar administration, it may be desirable to include an androgenic agent such as testosterone, dihydrotestosterone, testosterone analogues such as dehydroepiandrosterone (“DHEA”) and DHEA sulfate, or the like.

The pharmaceutical formulations used in the methods of the present invention may also include one or more pharmacologically active agents other than the estrogen and androgen. For example, the formulations may contain a vasodilating agent. Suitable vasodilating agents include, but are not limited to naturally occurring prostaglandins or hydrolyzable lower alkyl esters of a naturally occurring prostaglandin, as well as other vasodilators, such as, for example, sodium nitroprusside, diazenium diolates, molsidomine, linsidomine chlorhydrate, S-nitrosothiols, organic nitrates, pharmaceutically acceptable salts, esters, analogs, derivatives, prodrugs and inclusion complexes of any of the foregoing, and combinations thereof.

The pharmaceutical formulations used in the methods of the present invention may also include one or more lubricants. Suitable lubricants include, but are not limited to pullulan, ammonium poly(meth)acrylate, arabian gum, dextran, tamarindo gum, furcelleran, sodium starch-glycollic acid, sodium polyacrylate, hyaluronic acid, polyvinyl pyrrolidone, and the like and combinations thereof.

The pharmaceutical formulations used herein will typically contain one or more pharmaceutically acceptable carriers (also termed “excipients” or “vehicles”) suited to the particular type of formulation, i.e., gel, ointment, suppository, or the like. The vehicles are comprised of materials of naturally occurring or synthetic origin that do not adversely affect the estrogen, androgen, vasodilating agent or other components of the formulation. Suitable carriers for use herein include water, silicone, waxes, petroleum jelly, polyethylene glycol, propylene glycol, liposomes, sugars such as mannitol and lactose, and a variety of other materials, depending, again, on the specific type of formulation used.

The compositions used herein may be in the form of an ointment, cream, emulsion, lotion, gel, solid, sprays, solution, suspension, foam or liposomal composition; such formulations may be used for clitoral, vulvar or vaginal delivery. Alternatively, the compositions may be contained within a vaginal ring, tampon, suppository, sponge, pillow, puff, or osmotic pump system; these platforms are useful solely for vaginal delivery. Methods for preparing various dosage forms are known, or will be apparent, to those skilled in this art; for example, see Remington's Pharmaceutical Sciences, 18th Ed. (Easton, Pa.: Mack Publishing Company, 1990).

Ointments are semisolid preparations that are typically based on petrolatum or other petroleum derivatives. The specific ointment base to be used, as will be appreciated by those skilled in the art, is one that will provide for optimum drug delivery. As with other carriers or vehicles, an ointment base should be inert, stable, nonirritating and nonsensitizing. As explained in Remington: The Science and Practice of Pharmacy, supra, at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no welter and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be had to Remington: The Science and Practice of Pharmacy for further information.

Lotions are preparations that may be applied without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. It is generally necessary that the insoluble matter in a lotion be finely divided. Lotions will typically contain suspending agents to produce better dispersions as well as compounds useful for localizing the active agent in contact with the skin, e.g., methylcellulose, sodium carboxymethyl-cellulose, or the like.

Pharmaceutical emulsion formulations are generally formed from a dispersed phase (e.g., a pharmacologically active agent), a dispersion medium and an emulsifing agent. If desired, emulsion stabilizers can be included in the formulation as well. A number of pharmaceutically useful emulsions are known in the art, including oil-in-water (o/w) formulations, water-in-oil (w/o) formulations and multiple emulsions such as w/o/w or o/w/o formulations. Emulsifying agents suitable for use in such formulations include, but are not limited to, TWEEN 60®, Span 80®, cetostearyl alcohol, myristyl alcohol, glyceryl monostearate and sodium lauryl sulfate.

Pharmaceutical creams, as known in the art, are viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant.

A typical gel composition is formulated by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer to a solution. The preferred concentration of the gelling agent may range from 0.1 to 4 percent by weight of the total composition.

The compositions used herein can further comprise one or more additional ingredients, such as one or more thickening agents, medicinal agents or pharmaceuticals, bioadhesive polymers, inert carriers, lipid absorbents, viscosity stabilizers, chelating agents, buffers, anti-fading agents, stabilizers, moisture absorbents, fragrances, colorants, film-forming materials, and refatting agents, etc. One of skill in the art will readily be able to choose such additional excipients based on the physical and chemical properties desired in the final topical formulation. Of course, a single excipient may have multiple functions and properties.

Thickening agents are used to increase viscosity and improve bioadhesive properties. When present in a composition of the invention, the amount of thickening agent is preferably from about 1% to 10% by weight of the total composition weight, more preferably from about 2% to about 5% by weight.

The use of a tissue penetration enhancer is contemplated herein, and their use is employed to improve the permeability of an active ingredient into the vaginal tissue and not into the patient's blood stream. Penetration enhancers employed are those recognized in the art as safe for topical application to exposed tissue, for example one or more nonvolatile organic solvents such as, for example, amides, e.g., pyrrolidones; polyol ethers, e.g., glycol ethers; polyols, e.g., glycols, and derivatives thereof, etc.

In one embodiment of the invention, the amount of hormone administered to the vaginal tissue of the female is at least the minimum necessary to improve vaginal cell health as exhibited by increased numbers of vaginal cells (e.g., vaginal wall thickening), increased blood flow to vaginal tissue, and a generalized improvement of vaginal cell hypoxia and ischemia which may be the result of an illness, disease and/or medical treatment. As such, the aforementioned vaginal cell health effects are the desired results or therapeutic effects which provide for the regeneration of vaginal tissue.

In another embodiment of the present invention, a method of treating vaginal cell hypoxia in a female patient is provided wherein an evaluation of the condition of the vaginal tissue during the course of treatment is made to determine whether or not the treatment is necessarily continued.

In yet another embodiment of the invention, an effective amount of the hormone should be administered at least once a week to the vaginal tissue of the patient to improve vaginal cell health as exhibited by increased numbers of vaginal cells, increased blood flow to vaginal tissue, and a generalized improvement of vaginal cell hypoxia and ischemia. In still another embodiment of the invention, an effective amount of the active agent should be administered at least twice a week to the vaginal tissue of the patient to improve vaginal cell health as exhibited by increased numbers of vaginal cells, increased blood flow to vaginal tissue, and a generalized improvement of vaginal cell hypoxia and ischemia.

The methods of the present invention for promoting improved vaginal cell health as exhibited by increased numbers of vaginal cells, increased blood flow to vaginal tissue, and a generalized improvement of vaginal cell hypoxia and ischemia is accomplished by a non-systemic amount of at least one hormone selected from the group of estrogen, androgen, and pharmaceutically acceptable salt, ester or prodrug wherein the hormone does not enter the patient's blood stream in any substantial amount.

In yet another embodiment of the present invention, a method of promoting vaginal cell health by administering a pharmaceutical formulation containing a selected estrogen and/or androgen agent, in combination with a pharmaceutically acceptable vehicle.

In an embodiment of the present invention, a method of treating vaginal tissue is provided for a human female undergoing a therapy or treatment or having undergone a therapy or treatment that impairs the health of vaginal tissue cells and who is not currently receiving chronic hormonal therapy. The method comprises administering an effective, non-systemic dosage amount of a pharmaceutical formulation comprising at least one hormone selected from the group consisting of an estrogen and androgen or a pharmaceutically acceptable salt, ester, or pro-drug thereof, directly to the vaginal tissue of the female patient, at least once a week. This method of treating vaginal tissue alleviates the symptoms of vaginal cell hypoxia and/or atrophy caused by the chemotherapy or radiation therapy by increasing the flow of blood and nutrients to vaginal tissue cells

In another embodiment of the present invention, a method of treating vaginal tissue in a human female exhibiting clinical signs of a disease or condition that impairs the health of vaginal tissue cells and who is not receiving chronic hormonal therapy is provided. The method comprises administering an effective, non-systemic dosage amount of a pharmaceutical formulation comprising at least one member selected from the group consisting of an estrogen and androgen or a pharmaceutically acceptable salt, ester, or pro-drug thereof, directly to the vaginal tissue of the female patient, at least once in a seven day period. This method of treating vaginal tissue alleviates the symptoms of vaginal cell hypoxia and/or atrophy caused by diabetes or diseases and conditions that adversely affect vaginal tissue cells by increasing the flow of blood and nutrients to the vaginal tissue cells.

The methods of treating vaginal tissue, as contemplated herein, requires the administration of estrogen and analogues thereof at a level sufficient to exceed naturally occurring levels at the point of administration. For example, an amount of estrogen having estrogenic activity equivalent to from about 0.01 micrograms to about 100 micrograms ethinyl estradiol is applied directly to the vaginal tissue. In one embodiment of the invention, estrogen having estrogenic activity equivalent to from about 5 micrograms to about 50 micrograms ethinyl estradiol is applied directly to the vaginal tissue. In another embodiment of the invention, estrogen having estrogenic activity equivalent to from about 10 micrograms to about 35 micrograms ethinyl estradiol is applied directly to the vaginal tissue at least once a week. In yet another embodiment of the invention, estrogen having estrogenic activity equivalent to from about 0.1 micrograms to about 35 micrograms ethinyl estradiol is applied directly to the vaginal tissue twice a week.

The methods of treating vaginal tissue, as contemplated herein, requires the administration of androgen and analogues thereof in an amount having androgenic activity equivalent to from about 0.001 milligrams to about 3.00 milligrams methyltestosterone. In one embodiment of the invention, androgen having androgenic activity equivalent from about 0.01 milligrams to about 2.0 milligrams methyltestosterone is applied directly to the vaginal tissue. In another embodiment of the invention, androgen having androgenic activity equivalent to from about 0.1 milligrams to about 1.5 milligrams is applied directly to the vaginal tissue at least once a week. In yet another embodiment of the invention, androgen having androgenic activity equivalent to from about 0.1 milligrams to about 1.5 milligrams is applied directly to the vaginal tissue twice a week.

In another embodiment of the present invention the pharmaceutical formulation further comprises an effective amount of a vasodilating agent to promote regeneration of vaginal tissue.

The use one or more vasodilator agents, as contemplated herein, are administered in a dosage that is at least the minimum necessary to treat the vaginal tissue hypoxia.

In still another embodiment of the present invention, the claimed method further comprises the use of a water-soluble lubricant, and pharmaceutical formulations useful in conjunction with the aforementioned methods are provided.

It is to be understood that while the invention has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

Experimental: Topical estrogen and/or androgen cream, suppository, ointment and gel formulations are prepared:

### EXAMPLE 1

### Vaginal Cream

One application unit is equivalent to one (1) gram of cream. The application unit comprises 5 mcg of ethenyl estradiol and 0.5 mg of methyl testosterone.
One gram of cream has the following composition:

| | | |
|---|---|---|
| ethinyl estradiol | 5 | mcg |
| methyl testosterone | 0.5 | mg |
| Sorbitan monostearate | 45.0 | mg |
| Polysorbate 60 (Tween 60) | 15.0 | mg |
| Cetyl palmitate (Cutina CP-A) | 30.0 | mg |
| Viscous paraffin | 130.46 | mg |
| Cetylstearyl alcohol | 100.0 | mg |
| Hyaluronic acid | 50.0 | mg |
| Purified water | 630.0 | mg |

### EXAMPLE 2

### Suppository

Prepared by fusion or melt molding wherein one suppository unit comprises 15 mcg of ethenyl estradiol and 1.0 mg of methyl testosterone.
Per suppository the following components are combined:

| | | |
|---|---|---|
| ethinyl estradiol | 15 | mcg |
| methyl testosterone | 1.0 | mg |
| Polyethylene glycol 1000 | 1.700 | g |
| Polyethylene glycol 4000 | 0.070 | g |

### EXAMPLE 3

### Vaginal Ointment

One application unit is equivalent to one (1) gram of ointment. The application unit comprises 25 mcg of ethenyl estradiol and 0.5 mg of methyl testosterone.
One gram of ointment has the following composition:

| | | |
|---|---|---|
| ethinyl estradiol | 25.00 | mcg |
| methyl testosterone | 1.00 | mg |
| N-Methyl Pyrolidone (Pharmasolve) | 50.00 | mg |
| White Wax (White Bees Wax) | 67.50 | mg |
| Mineral Oil (Liquid Paraffin) | 144.00 | mg |
| Paraffin (Hard Paraffin) | 30.00 | mg |
| Petrolatum, White | 707.00 | mg |

### EXAMPLE 4

### Vaginal Gel

One application unit is equivalent to one (1) gram of gel. The application unit comprises 25 mcg of ethenyl estradiol and 0.5 mg of methyl testosterone.

| | | |
|---|---|---|
| ethinyl estradiol | 25 | mcg |
| methyl testosterone | 0.5 | mg |
| Hydroxypropylmethylcellulose (HPMC) | 0.5 | g |
| Carbopol RTM 934 | 0.25 | g |
| Glycofurol 75 | 0.2 | g |
| Aqua purificata | 30 | ml |

Obviously, other modifications and variations of the present invention are possible in light of the above teachings. It is, therefore, to be understood that changes may be made in the particular embodiments described above which run within the full intended scope of the invention.

1. A method of treating a human female exhibiting vaginal cell hypoxia and who is not currently receiving chronic hormonal therapy, the method comprising topically administering to the vaginal tissue of the female patient at least once in a seven day period a non-systemic vaginal cell hypoxia treatment amount of a composition comprising at least one hormone selected from the group consisting of estrogen and androgen and/or pharmaceutically acceptable salt and ester thereof.

2. The method of claim 1 wherein the estrogen is selected from the group consisting of 17 β-estradiol, estrone, estriol, ethinyl estradiol, estropipate, equilin, Δ8,9-dehyroestrone, 17α-estradiol, 17α-dihydroequilin, 17β-dihydroequilin, 17β-estradiol, equilenin, 17α-dihydroequilin, and 17β-dihydroequilenin, and combinations thereof.

3. (canceled)

4. The method of claim 1 wherein the androgen is selected from the group consisting of testosterone, dihydro-testosterone (DHT), methyltestosterone, dehydroepiandrostenedione (DHEA), and pharmaceutically acceptable salts, esters and pro-drugs thereof.

5. The method of claim 1 further comprising an effective amount of a vasodilator agent.

6. The method of claim 5 wherein the vasodilator is selected from the group consisting of prostaglandins, hydrolyzable lower alkyl esters of a naturally occurring prostaglandin, sodium nitroprusside, diazenium diolates, molsidomine, linsidomine chlorhydrate, S-nitrosothiols, organic nitrates, pharmaceutically acceptable salts, esters, analogs, derivatives, and prodrugs, and combinations thereof.

7. The method of claim 1 wherein the composition further comprises a pharmaceutically acceptable dosage form selected from the group consisting of ointment, cream, gel, lotion, solution, paste, bioadhesive, suppository or combination thereof.

8. The method of claim 1 further comprising evaluating the condition of the vaginal tissue during the course of treatment to determine the future course of said treatment.

9. The method of claim 7 wherein the dosage form further comprises a water-soluble lubricant.

10. The method of claim 7 wherein the dosage form further comprises a tissue penetration enhancer.

11. The method of claim 10 wherein the tissue penetration enhancer is a nonvolatile organic solvent selected from the group consisting of amides, poly ethers, polyols and mixtures thereof.

12. The method of claim 11 wherein the tissue penetration enhancer is selected from the group consisting of pyrrolidones, glycol ethers, glycols, and mixtures thereof.

13. The method of claim 1 wherein the estrogen is administered in a dosage amount having estrogenic activity equivalent to from about 0.01 micrograms to about 100 micrograms ethinyl estradiol.

14. The method of claim 13 wherein the estrogen is administered in a dosage amount having estrogenic activity equivalent to from about 5 micrograms to about 50 micrograms ethinyl estradiol.

15. The method of claim 14 wherein the estrogen is administered in a dosage amount having estrogenic activity equivalent to from about 10 micrograms to about 35 micrograms ethinyl estradiol.

16. The method of claim 1 wherein the androgen is administered in a dosage amount having androgenic activity equivalent to from about 0.001 milligrams to about 3.00 milligrams methyl testosterone.

17. The method of claim 16 wherein the androgen is administered in a dosage amount having androgenic activity equivalent to from about 0.01 milligrams to about 2.00 milligrams methyl testosterone.

18. The method of claim 17 wherein the androgen is administered in a dosage amount having androgenic activity equivalent to from about 0.1 milligrams to about 1.50 milligrams methyl testosterone.

19. The method of claim 1 comprising administering directly to the vaginal tissue of the female patient, at least twice in a seven day period.

20. The method of claim 19 comprising administering directly to the vaginal tissue of the female patient daily.

21. The method of claim 9 wherein the water soluble lubricant is selected from the group consisting of pullulan, ammonium poly(meth)acrylate, arabian gum, dextran, tamarindo gum, furcelleran, sodium starch-glycollic acid, sodium polyacrylate, hyaluronic acid and polyvinyl pyrrolidone and mixtures thereof.

22-68. (canceled)
